(19) United States
(12) Patent Application Publication
(10) Pub. No.: US 2005/0175683 A1
(43) Pub. Date: Aug. 11, 2005

(54) Preparation of lipid particles

(76) Inventor: Yuanpeng Zhang, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/970,861

(22) Filed: Oct. 22, 2004

Related U.S. Application Data
(60) Provisional application No. 60/514,451, filed on Oct. 24, 2003.

(51) Int. Cl. A61K 31/704; A61K 9/127

(52) U.S. Cl. 424/450; 514/34

(57) ABSTRACT

A method for preparing lipid particles comprising producing discrete droplets of vesicle-forming lipids in a solvent, where the droplets have a diameter and a volume, introducing the discrete droplets into an aqueous solution to form lipid particles suitable for in vivo administration. The droplet may further contain any one or more of oils, surfactants, targeting ligands, markers, or therapeutic and diagnostic agents. The droplets may be generated by a system selected from a nebulizer, an atomizer, a venturi mist generator, a focused acoustic ejector, and an electrospray device. This method can be used to select or regulate the size and/or size distribution of the lipid particles.

This application claims the benefit of U.S. Provisional Application No. 60/514,451 filed Oct. 24, 2003, which is incorporated herein by reference in its entirety.

### FIELD OF THE INVENTION

This invention relates generally to a simple, cost effective method for preparing lipid particles and particulates for delivery of therapeutic agents.

### BACKGROUND OF THE INVENTION

Many types of micro- and nano-particulate systems have been utilized as components of lipid particles for pharmaceutical agents. For example, liposomes, lipospheres, emulsomes, niosomes, emulsions, to name the most common examples, are particularly useful as lipid particles for both poorly water soluble or hydrophobic drugs and hydrophilic drugs. These lipid particles have the potential for providing controlled “depot” release of an administered drug over an extended time period, and of reducing the side effects of the drug, by limiting the concentration of free drug in the bloodstream.

The most widely used of these microparticulate systems is liposomes, which exist predominantly in the form of single unilamellar vesicles (SUVs, generally 20-500 nm in diameter and consisting of a single bilayer of phospholipids or other vesicle forming lipids), or multilamellar vesicles (MLVs, up to several microns in diameter and consisting of multiple bilayers entrapped onion-like within each other). Liposomes provide the potential for delivering solvated hydrophobic drugs and oils, as well as entrapped drugs or nucleic acids within the aqueous interior.

These advantages of liposome drug delivery apply to a variety of routes of administration, including intravenous, intramuscular, and subcutaneous, application to mucosal tissue, or delivery by inhalation. Where liposomes are administered by intravenous delivery, liposomes provide a further advantage of altering the tissue distribution of the drug. A review of liposome drug delivery systems is presented by Pozansky et al. (Pharm. Revs., 36(4):277) and Gregoriadis (Liposomes, Vol. III, 1984).

Generally, the optimal liposome size for use in parenteral administration is between about 50 nm and 200 nm. Liposomes in this size range can be sized by passage through conventional filters having a particle size discrimination of about 200 nm. This size range of liposomes favors biodistribution in certain target organs, such as tumor tissue, liver, spleen, and bone marrow, and gives more uniform and predictable drug-release rates and stability in the bloodstream. Liposomes whose sizes are less than about 300 nm also show less tendency to agglutinate during storage, and are thus generally safer and less toxic in parenteral use than larger-size liposomes. Uniform-size liposomes in a selected size range less than about 150 nm are also useful in many therapeutic applications. For example, because of their small size, SUVs are useful in targeting to tumor tissue or to hepatocyte cells, because of their ability to penetrate the endothelial lining of capillaries. SUVs are also advantageous in ophthalmic liposome formulations, because of the greater optical clarity of the smaller liposomes.

Liposomes are typically made by mixing vesicle-forming lipids with an aqueous buffer. Typically, a heterodisperse distribution of liposomes is obtained, having a size predominantly greater than about 1 micron (1,000 nm). These initial heterodispersed suspensions can be reduced in size and the size distribution narrowed by a number of known methods. Liposomes are typically sized by extrusion through progressively smaller pores, by sonication or homogenization, by detergent dialysis, or by solvent injection or evaporation.

Other lipid particles are also made using similar procedures. For example, lipospheres, emulsions, niosomes and emulsomes can all be generated using sonication. In a similar manner, U.S. Pat. No. 4,622,219 to Haynes describes a method of making a local anesthetic formulation by sonicating microdroplets of methoxyflurane in aqueous solution, and coating the methoxyflurane droplets with a monolayer of lipid molecules. However, this approach does not result in a liposphere, or liposomal lipid particle, and no liposomal formulation is discussed.

One size-processing method which is suitable for large-scale production is homogenization. Here, an initial heterodispersed liposome preparation is pumped under high pressure through a small orifice or reaction tank. The suspension is usually cycled through the reaction tank until a desired average size of liposome particles is achieved. A limitation of this method is that the liposome size distribution is typically quite broad and variable, depending on a number of process variables, such as pressure, the number of homogenization cycles, and internal temperature. Also, the processed fluid tends to pick up metal and oil contaminants from the homogenizer pump, and may be further contaminated by residual chemical agents used to sterilize the pump seals.

Sonication, or ultrasonic irradiation, of lipid dispersions, is another method that is used for reducing liposome sizes by shearing, and is especially useful for preparing SUVs. The processing capacity of this method is quite limited, since long-term sonication of relatively small volumes is required. Also, localized heat build-up during sonication can lead to oxidative damage to the lipids, and sonic probes shed titanium particles which are potentially quite toxic in vivo.

Another method known in the art is based on liposome extrusion through uniform pore-size polycarbonate membranes (Szoka, F., et al, (1978) Proc. Nat. Acad. Sci. (USA) 75:4194). This procedure has advantages over homogenization and sonication methods in that several membrane pore sizes are available for producing liposomes in different selected size ranges. In addition, the size distribution of the liposomes can be made quite narrow, particularly by cycling the material through the selected-size filter several times. Nonetheless, the membrane extrusion method has limitations in large-scale processing, including problems of membrane clogging, membrane fragility, and relatively slow throughput.

A further method of preparing liposomes is described in co-owned U.S. Pat. No. 4,737,323. This patent describes a liposome sizing method in which heterogeneous-size liposomes are sized by extrusion through an asymmetric ceramic filter. This method allows greater throughput rates, and avoids problems of clogging since high extrusion pressure and reverse-direction flow can be employed. However, like the membrane extrusion method, the filter-extrusion method requires post-liposome formation sizing. Further, the method may be limited where uniform-size SUVs are desired.

An alternative method for preparing liposomes is described in co-owned U.S. Pat. No. 5,000,887, which describes a method of forming liposomes having a uniform size distribution of about 300 nm or less. According to the method described in this patent, vesicle-forming lipids are dissolved in a water-miscible solvent, such as ethanol, and aqueous medium is added to a water:solvent ratio at which lipid assembly first occurs. The water:solvent ratio is then raised, under conditions which maintain the volume of the mixture substantially constant, until uniform-size liposomes are formed. The average size of the liposomes can be selectively varied by changing the ionic strength and lipid composition of the mixture. However, in this method, liposomes formed from neutral lipids have a size distribution in the range of 300 nm. In order to form smaller liposomes, charged lipids must be incorporated into the liposomes, or post-liposome formation sizing must be performed.

Additionally, PCT Publication No. WO 95/01777 describes a process for producing liposomes wherein the final liposome size is reported to be determined by the final proportion of ethanol in the formulation. The method may result in a liposome suspension containing large amounts of ethanol, which would require removal before use in a pharmaceutical formulation. In addition, this approach was not shown to be broadly applicable to different types of lipids or to produce liposomes having a desired size distribution.

Methods of forming liposomes are reviewed in greater detail by Y. P. Zhang, et al., Liposomes in Drug Delivery, in Polymeric Biomaterials, 2^{nd} edition, S. Dumitriu, Ed., Marcel Dekker, Inc., New York (2001). Generally, these methods provide heterogeneous sizes, are labor or cost intensive or require additional steps to remove residual solvent, detergent, or large liposomes. In none of the methods mentioned above, are liposomes or other lipid particles with a narrow, controllable and symmetrical size distribution produced by cost effective and labor saving procedures. Similarly, none of these methods are able to produce narrow and controllable sizes of lipospheres or emulsomes. Further, the methods known in the art require numerous additional steps to prepare lipid particles of a desired size and content, such as extrusion steps, dialysis and the like.

The invention addresses these deficiencies in the art by providing novel methods and devices for preparing lipid particles such as liposomes, lipospheres, emulsomes, niosomes, and emulsions.

### SUMMARY OF THE INVENTION

In one aspect, the invention comprises preparing lipid particles comprising producing discrete droplets of vesicle-forming lipids in a solvent. The droplets are introduced to an aqueous solution to form lipid particles. In one preferred embodiment, the lipid particles are suitable for in vivo administration.

In varying embodiments, the lipid particles may be liposomes, lipospheres, emulsomes, emulsions, niosomes, nanoparticles, and/or microparticles.

In one embodiment, the droplet volume is between about 10⁻⁴ fL and about 1 nL. In another embodiment, the droplet volume is between about 10⁻² fL and about 10 pL.

The lipid may be at least one of distearoyl phosphatidyl choline, distearoyl phosphatidyl ethanolamine, and hydrogenated soy phosphatidyl choline, or any other suitable vesicle-forming lipid. It will be appreciated that the lipid may include a combination of vesicle-forming lipids as well as a combination of vesicle-forming and non vesicle-forming lipids. In further embodiments, the solvent may include at least one of a cationic lipid, an anionic lipid, or a neutral-cationic lipid.

In an embodiment, the concentration of lipid in each droplet is between about 0.1 mg/mL up to and including the amount of lipid that is soluble in the particular solvent. In a further embodiment, the concentration of lipid in each droplet is between about 0.1 mg/mL and about 1 g/mL. In yet another embodiment, the concentration of lipid in each droplet is between about 1 mg/mL and about 100 mg/mL.

In an additional embodiment, at least one therapeutic agent is included in at least one of the solvent or the aqueous solution. In one embodiment, at least one the therapeutic agent is included in both the solvent and the aqueous solution. In yet another embodiment, at least a first therapeutic agent is included in the solvent and at least a second therapeutic agent is included in the aqueous solution. In one embodiment, the therapeutic agent is a chemotherapeutic agent, an anti-cancer agent, or antiviral agent. In a specific embodiment, the therapeutic agent is an anthracycline antibiotic. Exemplary anthracycline antibiotics include daunorubicin, doxorubicin, mitoxantrone, and bisantrene.

In a further embodiment, the solvent can include one or more of lipopolymers, targeting ligands, oils, surfactants, markers, and pharmaceutical excipients. In one embodiment, the lipopolymer is selected from polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, and polyaspartamide. In one of the preferred embodiments, the lipopolymer is polyethylene glycol. In yet another of the preferred embodiments, the lipopolymer includes polyethylene glycol chains having a molecular weight of between about 500 Daltons and about 10,000 Daltons.

In another embodiment, at least one ligand is attached to the distal end of at least a portion of the lipopolymers. In yet another embodiment, at least one ligand is attached the polar head group of at least a portion of the vesicle-forming lipids. It will be appreciated that at least a portion of both the lipopolymers and the vesicle-forming lipids may include an attached ligand. It will further be appreciated that different ligands may be used for attachment to the lipopolymer or the vesicle-forming lipids. Where non vesicle-forming lipids are included in the solvent, at least a portion of the non vesicle-forming lipids may include an attached ligand.

In one embodiment, the droplets are generated by a system selected from the group consisting of a nebulizer, an atomizer, a venturi mist generator, a focused acoustic ejector, and an electrospray device. Where the droplets are formed by an acoustic ejector, the droplets may be formed by applying a focused acoustic radiation at a focal point near a surface of the solution prior to and/or during introducing the droplets to the aqueous solution. The ejector may include a plurality of ejectors such that a plurality of droplets can be ejected from one or more reservoirs. In a further embodiment, the discrete droplets are produced as a mist and the mist of droplets are directed into contact with the aqueous solution.

### BRIEF DESCRIPTION OF THE DRAWINGS

FIG. 1 illustrates a schematic view of a method for preparing lipid particles.

FIG. 2 illustrates a schematic view of an embodiment where a focused acoustic ejector is coupled to a reservoir of lipid in solvent for introducing lipid/solvent droplets into an aqueous solution.

FIG. 3 illustrates a schematic view of an embodiment where nebulized lipid/solvent droplets are introduced into an aqueous solution.

### DETAILED DESCRIPTION OF THE INVENTION

### I. DEFINITIONS AND OVERVIEW

Unless otherwise indicated, the present invention is not limited to specific lipids, droplet generation techniques and/or droplet introduction technologies. It will be appreciated that atomizers, nebulizers, focused acoustic ejection devices, or the like, as such may vary. It should also be understood that the terminology used herein is for the purpose of describing particular embodiments only and is not intended to limit the scope of the present invention.

It should be noted that as used herein the singular forms “a,” “and” and “the” include plural referents unless the context clearly dictates otherwise. Thus, for example, it will be appreciated that reference to “a solvent” includes two or more solvents; reference to “a pharmaceutical agent” includes two or more pharmaceutical agents, and so forth.

Where a range of values is provided, it should be understood that each intervening value, to the tenth of the unit of the lower limit between the upper and lower limit of that range, and any other stated or intervening value in that stated range, is encompassed within the invention, unless the context clearly dictates otherwise. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The term “lipidic structure” or “lipid particle” is used herein to refer to the structure or particles formed by lipids in an aqueous solution as exemplified by liposomes, lipospheres, emulsomes, niosomes, emulsions, and the like.

The term “therapeutic agent” as used herein refers generally to a pharmaceutical, therapeutic, or diagnostic agent for administration to an animal, including a human. As used herein, the terms “therapeutic agent”, “compound,” and “drug” are used interchangeably

The term “hydrophobic substance” as used herein refers generally to a substance having solubility in water below about 0.1 mg/ml. A hydrophobic substance is not necessarily a drug, or even a compound per se, and can include mixtures of substances, natural product extracts, nanomaterials (e.g., fullerenes, carbon nanotubes, and gold nanoparticles), industrial products, and the like.

The term “amphipathic lipids” refers to lipids having both hydrophobic and hydrophilic regions, and includes liposome forming lipids as well as surfactant molecules such as lysolipids having only one hydrocarbon chain as exemplified by lysophosphatidylcholine.

“Vesicle-forming lipids” refers to amphipathic lipids which have hydrophobic and polar head group moieties, and which can form spontaneously into bilayer vesicles in water, as exemplified by phospholipids, or are stably incorporated into lipid bilayers, with the hydrophobic moiety in contact with the interior, hydrophobic region of the bilayer membrane, and the polar head group moiety oriented toward the exterior, polar surface of the membrane. The vesicle-forming lipids of this type typically include one or two hydrophobic acyl hydrocarbon chains or a steroid group, and may contain a chemically reactive group, such as an amine, acid, ester, aldehyde or alcohol, at the polar head group. Included in this class are the phospholipids, such as phosphatidyl choline (PC), phosphatidyl ethanolamine (PE), phosphatidic acid (PA), phosphatidyl inositol (PI), and sphingomyelin (SM), where the two hydrocarbon chains are typically between about 14-22 carbon atoms in length, and have varying degrees of unsaturation. Also included within the scope of the term “vesicle-forming lipids” are glycolipids, such as cerebrosides and gangliosides.

As used herein, the term “size distribution” or “particle size distribution” refers to the relative percentage by number of each of the different size fractions of the lipid particles.

For purposes of the invention, no distinction is made between the terms “nebulizer” and “atomizer,” and these terms are used interchangeably.

As used herein, the term “diagnostic” includes diagnostic tests for in vivo, in vitro or ex vivo applications to human and nonhuman patients, as well as imaging applications in medicine or other fields.

Abbreviations: PEG: polyethylene glycol; mPEG: methoxy-terminated polyethylene glycol; mPEG2000-DSPE: methoxy-terminated polyethylene glycol conjugated to phosphatidylethanolamine; Chol: cholesterol; PC: phosphatidylcholine; PHPC: partially hydrogenated phosphatidylcholine; PHEPC: partially hydrogenated egg phosphatidylcholine; PHSPC: partially hydrogenated soy phosphatidylcholine; DSPE: distearoyl phosphatidylethanolamine; POPC: palmitoyl oleyl phosphatidylcholine; HSPC: hydrogenated soy phosphatidylcholine.

### II. LIPID PARTICLES

In one aspect, the invention includes a method of forming lipid particles having a uniform and/or selected size distribution. Lipid particles are the structures or particles formed by introducing lipids into an aqueous solution. Lipid particles that can be formed using the methods described herein include liposomes; lipospheres; emulsomes; emulsions; niosomes; and nanoparticles and microparticles. The formulations of the lipid particles can include a wide variety of amphipathic lipids, oils, surfactants, markers, targeting ligands, lipopolymers, solvents, and the like. These components are discussed further below.

As discussed above, lipid particles find use particularly in formulations for delivery of therapeutic agents or drug delivery. Drug delivery using lipid particles is particularly useful to increase bioavailability, decrease toxicity, provide capabilities such as targeting, provide or enhance stealth to evade the body's natural defenses as exemplified by uptake by the reticular endothelial system (RES), enhance tissue or cell infiltration, provide controlled release of the drug, or combined functions of any of the above.

Liposomes are vesicles composed of one or more concentric lipid bilayers which contain an entrapped aqueous volume. The bilayers are composed of two lipid monolayers having a hydrophobic “tail” region and a hydrophilic “head” region, where the hydrophobic regions orient toward the center of the bilayer and the hydrophilic regions orient toward the inner or outer aqueous phase. Liposomes are generally grouped by size and/or whether they are unilamellar or multilamellar (MLVs). Small unilamellar vesicles (SUVs) are generally 20-500 nm in diameter. Generally the larger liposomes form MLVs, while the smaller liposomes are unilamellar, however, it will be appreciated that larger liposomes may be unilamellar and smaller liposomes may be multilamellar. In a preferred embodiment, the liposomes are SUVs or MLVs.

Lipospheres are generally spherical or nearly spherical structures formed by a single molecular layer of lipids molecules arranged about an oily core or oil droplet. Lipospheres provide a hydrophobic environment for hydrophobic substances sequestered away from contact with the aqueous phase.

Emulsomes consist of a hydrophobic core, such as oil, surrounded by one or more lipid bilayers. This construction allows for the creation of very small, stable particles.

Niosomes are structures similar to liposomes, where the niosomes include surfactant molecules, preferably nonionic surfactants, in addition to or in place of the lipid molecules.

Emulsions are macroscopic versions of lipospheres and emulsomes, with an inner core of either oil-in-water or water-in-oil. Specifically, emulsions are a mixture of the lipids and at least one aqueous liquid in which the lipids are present as droplets of microscopic or ultramicroscopic size distributed throughout the liquid. The lipids droplets may be formed in a bilayer surrounding an aqueous core as in liposomes or a single lipid layer surrounding an oily core as for lipospheres.

The size of the lipid particles can range widely in size, having a diameter from about 20 nm to about 1000 nm. In preferred embodiments, the lipid particles have a diameter from about 80 nm to about 200 nm. It will be appreciated that the size of the lipid particle can be selected according to the delivery route. For intravenous delivery, the lipid particles are sized from about 80 nm to about 200 nm, preferably from about 100 nm to about 175 nm, more preferably from about 90 nm to about 150 nm. For delivery by inhalation, the lipid particles are generally aerosolized or nebulized where the particle size is from about 1 μm to about 7 μm. For rapid drug absorption through alveolar membranes, the lipid particles are generally sized about 10 nm to about 100 nm. For subcutaneous delivery, the lipid particles are sized from about 100 nm to about 250 nm.

A. Lipids

The lipids included in the lipid formulations of the present invention are generally vesicle-forming lipids. The vesicle-forming lipids are preferably those having two hydrocarbon chains, typically acyl chains, and a polar head group. Included in this class are the phospholipids, such as phosphatidylcholine (PC), PE, phosphatidic acid (PA), phosphatidylinositol (PI), and sphingomyelin (SM), where the two hydrocarbon chains are typically between about 14-22 carbon atoms in length, and have varying degrees of unsaturation. Also included in this class are the glycolipids, such as cerebrosides and gangliosides. A preferred vesicle-forming lipid is a phospholipid. Another vesicle-forming lipid which may be employed includes cholesterol, cholesterol derivatives, such as cholesterol sulfate and cholesterol hemisuccinate, and related sterols.

More generally, the term “vesicle-forming lipid” is intended to include any amphipathic lipid having hydrophobic and polar head group moieties, and which (a) by itself can form spontaneously into bilayer vesicles in an aqueous medium, as exemplified by phospholipids, or (b) is stably incorporated into lipid bilayers in combination with phospholipids, with its hydrophobic moiety in contact with the interior, hydrophobic region of the bilayer membrane, and its polar head group moiety oriented toward the exterior, polar surface of the membrane.

In some instances, it may be desirable to include lipids having branched hydrocarbon chains. Mixtures of lipids, such as egg or soy phosphatidylcholine, having variable acyl chain composition, can be utilized in its partially hydrogenated state or natural state. In Example 1, partially hydrogenated soy phosphatidylcholine was utilized (PHSPC).

In a preferred embodiment, the vesicle-forming lipid is selected from one or more of distearoyl phosphatidyl choline (DSPC), distearoyl phosphatidyl ethanolamine (DSPE), and hydrogenated soy phosphatidyl choline (HSPC).

In other embodiments, the lipid particles may further include cationic and/or anionic lipids. Cationic lipids include the neutral cationic lipids as described in commonly owned U.S. patent Publication No. 20030031704A1, as well as cationic lipids such as dialkyl dimethyl ammonium bromides (e.g., dimethyldioctacylammonium bromide (DDAB)) and dialkyl trimethylammonium 1,2-dioleyl-3-trimethylammonium-propane (DOTAP). Numerous other examples of cationic lipids are discussed in the review by Y. P. Zhang, et al. (Liposomes in Drug Delivery, in Polymeric Biomaterials, 2^{nd} edition, S. Dumitriu, Ed., Marcel Dekker, Inc., New York (2001)).

Anionic lipids include, without limitation, the commonly used phosphatidylserine, phosphatidylinositol and phosphatidic acid, as well as gangliosides such as G_{M1}, and the like.

The lipids of the invention may be prepared using standard synthetic methods. The lipids of the invention are further commercially available (Avanti Polar Lipids, Inc., Birmingham, Ala.).

It will be appreciated that the lipid particles may include one or more different types of lipids. In one embodiment, the lipid particles may include two or more different types of amphipathic lipids and one or more non-amphipathic lipids. In one embodiment including two or more different types of lipids, the lipids are mixed such that the lipid particles can be prepared using a wide variety of lipids present in various mole fractions. For example, liposomes are commonly prepared from mixtures of PE, PC and cholesterol, as well as lidopolymers, discussed below.

B. Therapeutic Agents

A preferred embodiment of the present delivery vehicles is as a lipid particle for the delivery of therapeutic or diagnostic agents to human patients. Included are pharmaceutical, therapeutic or diagnostic agents for administration to a human or an animal, although other uses can be readily envisioned. Also included are prodrugs that can be converted after administration into an active form.

Therapeutic agents that can be used in the present formulations include hydrophilic drugs (i.e., having solubility in water at room temperature (25° C.) of greater than 0.01% (i.e., 0.1 mg/ml)) and hydrophobic drugs (i.e., having solubility in water at room temperature (25° C.) of less than 0.01%).

The therapeutic agent is typically entrapped in the lipid layer of the lipid particle. By “entrapped” it is meant that a therapeutic agent is entrapped in the liposome central compartment and/or lipid layer spaces, is associated with the external lipid surface, or is both entrapped internally and externally associated with the lipid particles. The therapeutic agent may be hydrophilic, hydrophobic, or amphipathic. Hydrophilic molecules typically are entrapped within the aqueous compartment of the lipid particle for liposomes or niosomes, in association with the surface of liposomes, niosomes, or emulsomes, or in the aqueous intrabilayer space of liposomes. Hydrophobic molecules are typically localized in the lipid layer, or the oil core, where present. Amphipathic molecules often localize in the lipid/aqueous interface.

In some instances, the hydrophilic substances present in the aqueous solution can associate with the surface of the lipid particle, such as by hydrophobic or electrostatic attraction. For example, polyanionic compounds will associate with cationic surface charges, or polycationic compounds will associate with anionic surface charges on the lipid particles, or other compounds will interact favorably with the interfacial layer provided by the lipid head groups present at the interface between lipid and aqueous phases.

Exemplary hydrophobic drugs include, without limitation, steroids, bryostatin-1, cephalomannine, cisplatin, plicamycin, resveratrol, camptothecins such as topotecan, and irinotecan; local anesthetics such as lidocaine or bupivicaine, anthracycline antibiotics such as daunorubicin, doxorubicin and idarubicin; epipodophyllotoxins such as etoposide and teniposide; taxanes such as paclitaxel and docetaxel; antifungal agents, including, but not limited to, the polyene antifungal agents such as amphotericins, partricins, nystatin; and analogs and derivatives of all of the above.

As stated above, the therapeutic agents may be a prodrug. Prodrugs include, without limitation, fluoropyrimidine and cytidine analogs, such as gemcitabine, capecitabine, 5-fluorocytosine, 5′-deoxy-5-fluorouridine; activated etoposides such as the 3,4-dihydroxyphenyl carbamate derivative of etoposide, VP-16, ProVP-16I and II; cyclophosphamide, irinotecan, mitomycin C, AQ4N, ganglicovir, Herpes simplex thymidine kinase, dinitrobenzamide, CMDA or ZP2767P with Pseudomonas aeruginosa carboxypeptidase, G(2) indole-3-acetic acid activated by horseradish peroxidase, prodrugs of camptothecin, such as 9-aminocamptothecin glucuronide, and soluble polymer carrier linked camptothecin (MAG-camptothecin; CB1954 activated by E. coli nitroreductase; and tributyrin.

In some embodiments, the therapeutic agent includes nucleic acids (e.g., DNA, RNA, ribozymes, antisense RNA, siRNA, vectors, genes, genomic fragments, nucleic acids comprising modified nucleotides or modified linkages), which can be entrapped upon formation of liposomes or associate with a lipid particle bearing a positive surface charge, such as provided by including cationic surfactants or cationic lipids in the formulation.

In other embodiments, the therapeutic agent is a cytotoxic drug. In yet another embodiment, the therapeutic agent is a vaccine. In another embodiment, peptides, saccharides, or other antigens, are covalently attached to the lipids or lipopolymers, discussed further below, of the lipid particles. Such lipid particles are effective as adjuvants for enhancing the immunogenic responses to exposed antigens on the surface of the lipid particles.

One skilled in the art will appreciate that the lipid particles and methods of preparing them described herein are not restricted to pharmaceutical agents. Thus the lipid particles described herein are useful in formulations for horticulture, such as fertilizers, pesticides, plant or fungal growth regulators or inhibitors; biotechnology, such as gene transfection agents, vectors, and markers (e.g., fluorophores, radiotracers, dyes, enzymes); in medicine for applications such as therapeutics, diagnostics and imaging; in nanotechnology such as for handling and delivering nanotubes or nanospheres, fullerenes, quantum dots, etc.; for industrial applications such as manufacturing thin films or polymerization within emulsions; in cosmetics and cosmeceuticals, such as formulating oils and essences, or skin care agents; as nutriceuticals for formulating vitamins and plant or fungal extracts, and the like.

C. Lipopolymers

In one embodiment, the lipid particles include at least one lipopolymer, a lipid derivatized with a polymer, preferably a vesicle-forming lipid derivatized with a hydrophilic polymer. Preparation of vesicle-forming lipids derivatized with hydrophilic polymers has been described, for example, in U.S. Pat. No. 5,213,804. In one embodiment, between 1-20 mole percent of the vesicle-forming lipids in the lipid layer are derivatized with a hydrophilic polymer.

Exemplary hydrophilic polymers include polyethyleneglycol, polyvinylpyrrolidone, polyvinylmethylether, polymethyloxazoline, polyethyloxazoline, polyhydroxypropyloxazoline, polyhydroxypropylmethacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, polyethyleneoxidepolypropylene oxide copolymers, copolymers of the above-recited polymers, and mixtures thereof. Properties and reactions with many of these polymers are described in U.S. Pat. Nos. 5,395,619 and 5,631,018. Other polymers which may be suitable include polylactic acid, polyglycolic acid, and copolymers thereof, as well as derivatized celluloses, such as hydroxymethylcellulose or hydroxyethylcellulose. Additionally, block copolymers or random copolymers of these polymers, particularly including PEG segments, may be suitable, as described in U.S. Pat. Nos. 5,395,619 and 5,631,018. Methods for preparing lipids derivatized with hydrophilic polymers, such as PEG, are well known e.g., as described in co-owned U.S. Pat. No. 5,013,556.

A preferred hydrophilic polymer chain is polyethyleneglycol (PEG), preferably a PEG chain having a molecular weight between 500-15,000 daltons, more preferably between 1,000 and 5,000 daltons. Methoxy or ethoxy-capped analogues of PEG are also preferred hydrophilic polymers, commercially available in a variety of polymer sizes, e.g., 120-20,000 daltons.

Additional hydrophilic polymers include polysaccharides, such as those described in U.S. patent application Publication Ser. No. 2003/0133972 to Danthi. Such polysaccharides include, but are not limited to, dextrans, glucans, mannans, fucans, glycogen, cellulose, starch, as well as other homo- or heteropolymers, and the like.

As has been described, for example in U.S. Pat. No. 5,213,804, including such a derivatized lipid in a lipid formulation forms a surface coating of hydrophilic polymer chains around the lipid particle. For liposomes, the surface coating of hydrophilic polymer chains has been shown to be effective to increase the in vivo blood circulation lifetime of the liposomes when compared to liposomes lacking such a coating. Additionally, including such a derivatized lipid offers greater flexibility in modulating interactions of the liposomal surface with target cells (conferring stealth capabilities) and with the RES (Miller et al., (1998) Biochemistry, 37:12875-12883).

PEG-substituted synthetic ceramides have been used as uncharged components of sterically stabilized liposomes (Webb et al., (1998) Biochim. Biophys. Acta, 1372:272-282); however, these molecules are complex and expensive to prepare, and they generally do not pack into the phospholipid bilayer as well as diacyl glycerophospholipids.

Lipopolymers including a neutral linkage in place of the charged phosphate linkage of PEG-phospholipids can also be used, as described in co-owned U.S. Pat. No. 6,586,001. The neutral linkage is typically selected from a carbamate, an ester, an amide, a carbonate, a urea, an amine, and an ether. Hydrolyzable or otherwise cleavable linkages, such as disulfides, hydrazones, peptides, carbonates, and esters, are preferred in applications where it is desirable to remove the PEG chains after a given circulation time in vivo. A preferred releasable linkage is a dithiobenzyl linkage, described in co-pending U.S. patent Publication No. 20030031704A1. This feature can be useful in releasing drug or facilitating uptake into cells after the liposome has reached its target (Martin et al., U.S. Pat. No. 5,891,468, and PCT Publication No. WO 98/18813 (1998)) or in temporarily masking a targeting ligand, discussed below.

D. Targeting Ligands

The lipid particles may optionally include surface groups, such as antibodies or antibody fragments, small effector molecules for interacting with cell-surface receptors, antigens, and other like compounds, for achieving desired target-binding properties to specific cell populations. Such ligands can be included in the lipid particles by including a lipid derivatized with the targeting molecule, or by including a lipid having a polar-head chemical group that can be derivatized with the targeting molecule. Alternatively, a targeting moiety can be inserted into the lipid particles after formation by incubating the lipid particles with a ligand-polymer-lipid conjugate.

Lipids can be derivatized with the targeting ligand by covalently attaching the ligand to the free distal end of a hydrophilic polymer chain, which is attached at its proximal end to a vesicle-forming lipid, and incorporating the targeting ligand into liposomes (Zalipsky, S., (1997) Bioconjugate Chem., 8(2):111-118). Alternatively, the targeting ligand can be derivatized to a lipid (e.g., phosphatidylethanolamine) directly or through a linking group, thereby remaining masked until removal of the hydrophilic polymer chains. Of course, it will be appreciated by one skilled in the art that it may be desired at times to incorporate the targeting ligand into the lipid particle without the presence of the lipopolymer.

There are a wide variety of techniques for attaching a selected hydrophilic polymer to a selected lipid and activating the free, unattached end of the polymer for reaction with a selected ligand, and in particular, the hydrophilic polymer polyethyleneglycol (PEG) has been widely studied (Zalipsky, S., (1997) Bioconjugate Chem., 8(2):111-118; Allen, T. M., et al., (1995) Biochemicia et Biophysica Acta 1237:99-108; Zalipsky, S., (1993) Bioconjugate Chem., 4(4):296-299; Zalipsky, S., et al., (1994) FEBS Lett. 353:71-74; Zalipsky, S., et al., (1995) Bioconjugate Chemistry, 705-708; Zalipsky, S., in STEALTH LIPOSOMES (D. Lasic and F. Martin, Eds.) Chapter 9, CRC Press, Boca Raton, Fla. (1995)).

As further described in the method section below, targeting ligands can be present in solvent including the lipids. Alternatively, the targeting ligand can be added to the liposome or other lipid particle after formation of the lipid particle, especially for targeting ligands that may be damaged by exposure to solvent (Zalipsky, S., (1997) Bioconjugate Chem., 8(2):111-118).

E. Oils

As noted above, the lipid particles may be formed to have an oil inner core. Particularly, oil may constitute the hydrocarbon component of lipospheres, emulsomes and emulsions. Oils suitable for use in the lipid particles include, without limitation, triglycerides, such as triolein, trilinolein, tricaprin, trinervonin, trinonadecanoin, trimyristin, trinonanoin, diglycerides, such as 1,3-distearin, 1,3-dipalmitin, monoglycerides, such as monoolein, and fatty acids, such as stearic acid, oleic acid or arachidonic acid, of animal or plant origin; synthetic oils; semi-synthetic oils; or hydrocarbons. Oils can also include silicon oils, such as described in U.S. Pat. No. 5,688,897 to Malick. Examples of silicon oils include polymethyldiphenyl siloxane, such as GE Silicone SF 1154 (General Electric, Waterford, N.Y.) or fluorosilicones PS 181 and PS 182. In one embodiment, the oil may itself be a therapeutic or diagnostic agent.

It will be appreciated that the proportion of oils and lipids is generally higher for lipospheres, emulsomes and emulsions. Generally, one quarter or more of the total formulation can be oil for these lipid particles. For lipospheres, generally up to ⅔ of the total formulation can be oil, and for emulsomes, generally about ⅓ of the total formulation can be oil.

F. Surfactants

In one embodiment, a surfactant can be included in the lipid particles described herein. Surfactants include ionic surfactants (possessing at least one ionized moiety) and nonionic surfactants (having no ionized groups). Ionic surfactants include, without limitation, anionic surfactants, such as fatty acids and salts of fatty acids (e.g., sodium lauryl sulfate); sterol acids and salts thereof (e.g., cholate and deoxycholate); cationic surfactants, such as alkyl tri-methyl and ethyl ammonium bromides (e.g., cetyl triethyl ammonium bromide (CTAB) and C₁₆TAB); amphoteric surfactants, such as lysolipids (e.g., lysophosphatidylcholine or phosphatidylethanolamine), and CHAPS; Zwittergents, such as Zwittergent® 3-14.

In another embodiment, nonionic surfactants are included in the lipid particles. Nonionic surfactants are particularly useful in the generation of niosomes, emulsomes and emulsions. Nonionic surfactants include, without limitation, fatty alcohols, that is, alcohols having the structural formula CH₃(CH₂)ₙC(H)OH (e.g., where n is at least 6), such as lauryl, cetyl and stearyl alcohols; fatty sugars, such as octyl glucoside and digitonin; Lubrols, such as Lubrol® PX; Tritons, such as TRITON® X-100; Nonidents, such as Nonident P-40; sorbitan fatty acid esters (such as those sold under the trade name SPAN®), polyoxyethylene sorbitan fatty acid esters (such as those sold under the trade name TWEEN®), polyoxyethylene fatty acid esters (such as those sold under the trade name MYRJ®), polyoxyethylene steroidal esters, polyoxypropylene sorbitan fatty acid esters, polyoxypropylene fatty acid esters, polyoxypropylene steroidal esters, polyoxyethylene ethers (such as those sold under the trade name BRIJ®), polyglycol ethers (such as those sold under the trade name TERGITOL®), and the like. Preferred nonionic surfactants for use as surfactants herein are polyglycol ethers, polyoxyethylene sorbitan trioleate, sorbitan monopalmitate, polysorbate 80, polyoxyethylene 4-lauryl ether, propylene glycol, and mixtures thereof.

Anionic surfactants which may be used as the solubilizing agent herein include long-chain alkyl sulfonates, carboxylates, and sulfates, as well as alkyl aryl sulfonates, and the like. Preferred anionic surfactants are sodium dodecyl sulfate, dialkyl sodium sulfosuccinate (e.g., sodium bis-(2-ethylhexyl)-sulfosuccinate), sodium 7-ethyl-2-methyl-4-dodecyl sulfate and sodium dodecylbenzene sulfonate. Cationic surfactants which may be used to solubilize the active agent are generally long-chain amine salts or quaternary ammonium salts, e.g., decyltrimethylammonium bromide, dodecyltrimethylammonium bromide, tetradecyltrimethylammonium bromide, tetradecyltrimethylammonium chloride, and the like. Amphoteric surfactants are generally, although not necessarily, compounds which include a carboxylate or phosphate group as the anion and an amino or quaternary ammonium moiety as the cation. These include, for example, various polypeptides, proteins, alkyl betaines, and natural phospholipids such as lysolecithins and lysocephalins.

In a preferred embodiment the surfactant is present in the range of approximately 1 to 50 mole percent relative to the amount of lipid in the particles, and more preferably in the range of approximately 1 to 25 mole percent. The maximum amount of surfactant depends on the surfactant and lipid composition, and preferably the surfactant is not present in an amount that disrupts the structure of the lipid particle. One skilled in the art will appreciate that the surfactant can be present at higher or lower mole fractions for desired purposes.

Where the lipid particles described herein are to be used in the administration of pharmaceutical agents, surfactants should be chosen according to pharmaceutical acceptability. For example, in the construction of a niosome for systemic administration of a pharmaceutical agent to a human patient, a nonionic surfactant such as TWEEN® 80 would be appropriate. It will be appreciated that one skilled in the art is aware of the formulation requirements for human and animal patients, and understands that the surfactants that are appropriate can vary depending on the use.

G. Markers

Markers can also be included in the lipid particles of the invention. Aqueous markers, such as dyes, radioactive tracers, and the like, preferably are present in the aqueous solution during formation of the lipid particles, discussed further below.

H. Administration

The lipid particles of the invention may be administered to the patient by a variety of different varying means depending upon the intended application. As one skilled in the art would recognize, administration of the lipid particles can be carried out in various fashions, for example, via topical administration, including, but not limited to, dermal, ocular and rectal; transdermal, via passive or active means, e.g., using a patch, a carrier, or iontophoresis; transmucosal, e.g., sublingual, buccal, rectal, vaginal, or transurethral; oral, e.g., gastric or duodenal; parenteral injection into a body cavity or vessel, e.g., intraperitoneal, intravenous, intralymphatic, intratumoral, intramuscular, interstitial, intraarterial, subcutaneous, intralesional, intraocular, intrasynovial, intraarticular; via inhalation, e.g., pulmonary or nasal inhalation, using e.g., a nebulizer. Preferably, the lipid particles are administered parenterally or intratumorally.

Where systemic administration is desired, the lipid particles should have a size small enough to circulate within the capillary network without occluding any vessels. Preferably, such lipid particles are between about 20 nm and 500 nm in diameter, more preferably 80-200 nm. For administration to interstitial tissues, the lipid particle should be small enough to penetrate the endothelial tissues (e.g., have diameters smaller than about 100 nm).

### III. METHOD OF FORMING LIPID PARTICLES

In one aspect, the invention provides a method for preparing lipid particles, including liposomes, lipospheres, emulsomes, niosomes, emulsions, and the like, comprising introducing a discrete droplet comprising vesicle-forming lipids in a solvent into an aqueous solution. The lipid particles are discussed hereafter are liposomes, however, it will be appreciated that the discussion applies to the other lipid particles.

Without being limited as to theory, it is believed that the size of the liposomes and other lipid particles formed is primarily controlled by the size of the droplets and the amount of lipid in each droplet, as well as by the solvent, surfactant and oil, if present, the concentration of lipid in the solvent, the aqueous conditions, rate of droplet generation, and the rate of the solvent dispersal in the aqueous solution. It is further believed that the size distribution of the liposomes or other lipid particles is primarily controlled by the distribution of the droplets. For example, droplets that are similar in size, lipid concentration, etc. will produce a substantially uniform or similar size distribution for the liposomes.

An advantage of the methods described herein is that the need for further sizing steps or dialysis is minimized or obviated, resulting in time and cost savings in manufacturing. In a preferred embodiment, the liposomes may be used for in vivo application without any further processing such as sizing. The methods described herein can also be used in conjunction with extrusion, sonication or other prior art methods for forming lipid particles.

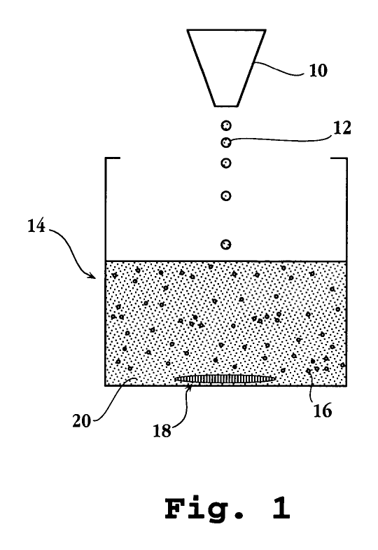

FIG. 1 depicts an embodiment of system for forming lipid particles 16 using a droplet generation system 10. As seen in FIG. 1, the lipid particles 16 are formed by introducing a droplet 12 of a solution comprising lipids in a solvent from the droplet generation system 10 into a collection vessel 14 containing an aqueous solution 20.

Vesicle-forming lipids as described above are dissolved in a suitable solvent. Solvents that can be used in the present methods include any solvent in which lipids are sufficiently soluble to achieve a minimal concentration of about 1 mM. The lipid concentration in the solvent is about 0.1 mg/mL to the maximum amount of lipid soluble in the solvent. It will be appreciated that this upper limit is determined by solubility of the lipid in the solvent. In a preferred embodiment, the lipid concentration in the solvent is between about 0.1 mg/mL and about 1 g/mL. Preferably, the lipid concentration in the solvent is between about 1 mg/mL to about 100 mg/mL.

On introduction of the droplets of solvent and lipid to the aqueous reservoir 14, a preferred solvent dissipates into the bulk aqueous phase or evaporates, allowing the lipids and other components to associate to form the lipid particle in the aqueous phase. The solvent can be water miscible or water immiscible, depending on the particular characteristics of the lipid formulation, solubility requirements of the therapeutic agent, and the lipid particle desired.

Suitable organic solvents include, without limitation, hydrocarbons, including aliphatic alkanes such as hexane, heptane, octane, etc., cyclic alkanes such as cyclohexane, and aromatic hydrocarbons such as benzene, cumene, pseudocumene, cymene, styrene, toluene, xylenes, tetrahydronaphthalene and mesitylene; halogenated hydrocarbons such as carbon tetrachloride, chloroform, bromoform, methyl chloroform, chlorobenzene, o-dichlorobenzene, chloroethane, 1,1-dichloroethane, dichloromethane, tetrachloroethanes, epichlorohydrin, trichloroethylene and tetrachloroethylene; ethers including alkyl ethers such as digthyl ether, diisopropyl ether, diisobutyl ether, dimethoxymethane, or cyclic ethers such as 1,4-dioxane, 1,3-dioxolane, furan, tetrahydropyran and tetrahydrofuran; aldehydes such as methyl formate, ethyl formate and furfural; ketones such as acetone, diisobutyl ketone, cyclohexanone, methyl ethyl ketone, N-methyl-2-pyrrolidone and isophorone; amides such as dimethyl formamide and dimethyl acetamide; alcohols such as ethanol, isopropanol, t-butyl alcohol, cyclohexanol, glycerol, ethylene glycol and propylene glycol; amines, including cyclic amines such as pyridine, piperidine, 2-methylpyridine, morpholine, etc., and mono-, di- and tri-substituted amines such as trimethylamine, dimethylamine, methylamine, triethylamine, diethylamine, ethylamine, n-butylamine, t-butylamine, triethanolamine, diethanolamine and ethanolamine, and amine-substituted hydrocarbons such as ethylene diamine, diethylene triamine; carboxylic acids such as acetic acid, trifluoroacetic acid and formic acid; esters such as ethyl acetate, isopentyl acetate, propylacetate, etc.; lactams such as caprolactam; nitriles such as acetonitrile, propane nitrile and adiponitrile; organic nitrates such as nitrobenzene, nitroethane and nitromethane; sulfides such as carbon disulfide; and sulfoxides such as dimethylsulfoxide.

Preferred solvents include alcohols such as ethanol, ethers such as diethyl ether, DMSO, and halogenated hydrocarbons such as chloroform and methylene chloride.

The solvent can further comprise at least one lipopolymer, one or more therapeutic agents, a lipid derivatized with a targeting ligand, a sterol, a cationic lipid, an anionic lipid, a surfactant, an oil, one or more markers, and the like. It will be appreciated that some of these components can be added to the aqueous solution after the liposomes are formed, such as the lipopolymer, therapeutic agent, and/or lipid derivatized with a targeting ligand. It will be appreciated that the solvent, the aqueous solution, or both can include the therapeutic or diagnostic agent or an excipient. When oil is present as a component of the lipid particle, it is preferably present in the droplet prior to introduction of the droplet to the aqueous solution.

Droplets 12 are generated from the lipid/solvent solution by any suitable means. Exemplary systems for generating the droplets include a nebulizer, an atomizer, a venturi mist generator, a focused acoustic ejector, an electrospray device, or the like, so long as the device or method of generating droplets provides droplets having the size ranges desired for preparing the lipid particles described herein. Preferably, the devices and methods for generating droplets provide droplets at a sufficient rate to prepare the lipid particle in a time and cost effective manner. Generation of droplets with a focused acoustic generator and a nebulizer are discussed further below.

These droplets are then introduced into an aqueous solvent 20 to form the lipid particles 16. The aqueous solution serves as the receptacle for the droplets comprising vesicle-forming lipids in solvent, along with other suitable components, as discussed above. Upon introduction of the droplets, the lipid particles form by diffusion or evaporation of the solvent (and in some instances, the surfactant) out of the droplet and into the aqueous phase, leaving the lipids, oils, surfactants etc. to form structures according to the composition of the droplet. It will be appreciated that the temperature, electrolytes and electrolyte concentration, pressure and the like can all be adjusted to affect the structures formed. Generally, the aqueous solution should be maintained at a temperature above the main phase transition of the lipids being introduced into the aqueous phase.

The aqueous solution generally comprises water, with optional solutes as desired. Optional solutes can include, without limitation, electrolytes, proteins, peptides, sugars, chaotropic agents, chelating agents, anti-oxidants (e.g., ascorbic acid, sodium ascorbate, vitamin E); acid, neutral or basic buffers (e.g., mono- or bi-basic phosphates); bacteriostats, and aqueous and non-aqueous sterile suspensions that can include suspending agents, solubilizers, thickening agents, stabilizers, pharmaceutical excipients, and preservatives (e.g., alkyl paraben, benzyl alcohol); ionic or non-ionic surfactants, as discussed above, polysorbates, co-solvents; polyalcohols such as i.e. glycerol, mannitol and xylitol. The aqueous solution can also be pre-equilibrated with a submicellar concentration of amphiphilic lipids (e.g., about 1 μM concentration of lipids) or surfactant, as desired.

The aqueous solution can contain any solute or cosolvent that it is desired to entrap or associate with the hydrophilic surfaces or the hydrophobic interior of the lipid particles. Thus the aqueous solution can further contain at least one therapeutic agent to be entrapped. By “entrapped” it is meant that the therapeutic agent is entrapped in the lipid particle central compartment and/or lipid bilayer spaces, is associated with the external lipid particle surface, or is both entrapped internally and externally associated with the lipid particles. The therapeutic agent may be hydrophilic, hydrophobic, or amphipathic. Hydrophilic molecules typically are entrapped within the aqueous compartment of the lipid particle or in the aqueous intrabilayer space of liposomes. Hydrophobic molecules are typically localized in either the inner or external bilayer core of liposomes, are entrapped within the oil core, or are associated with the non-polar head group of the lipid. Amphipathic molecules often localize in the lipid/aqueous interface.

In another embodiment, the aqueous solution may include solutes that associate with the surface of the lipid particles, including nucleic acids or other polymers.

For pharmaceutical formulations, typically the aqueous solution contains solutes that render the formulation isotonic with the blood of the intended recipient. Typically pharmaceutical formulations may contain one or more pharmaceutically acceptable electrolytes such as NaCl, KCl, MgSO₄, and CaCl₂; sugars including glucose and sucrose; and/or cryoprotectants such as glycerol, trehalose, and mannitose. Additionally, the aqueous solution may include aqueous markers such as dyes, radioactive tracers, and water soluble fluorophores, such as carboxyfluorescein. For liposomes, a portion of the aqueous solution is encapsulated inside the liposomes forming the aqueous core of the liposome.

One skilled in the art will appreciate that the pH can be adjusted for optimum performance of the particular lipids, oils and surfactants, etc., present in the droplets, which will depend on the intended use of the formulation. Typically, the pH in the aqueous solution will be in the range from about 3 to about 8 for most purposes in medicine, horticulture, biotechnology, and cosmetics and cosmeceuticals. However, any pH can be used for forming the lipid particles, so long as the components are stable at that pH. The pH can be adjusted to a more neutral, basic or acidic pH after formation of the lipid particle. For systemic drug delivery, a physiologically acceptable pH is generally desired, typically a pH of about 7.4. The lipid particles can also be formed in aqueous solution at low or high pH, and the pH adjusted to the desired range afterwards.

For remote loading of agents, pharmaceutical or otherwise, the liposomal delivery vehicle can be prepared in an aqueous solution containing ammonium sulfate, and then transferred to an aqueous solution having a lower concentration of ammonium sulfate (e.g., using dialysis or chromatography), providing a pH gradient driving the encapsulation of a later added drug. Remote loading has been described in detail in U.S. Pat. No. 5,192,549 to Barenholz, and in U.S. Pat. No. 6,465,008 to Slater (particularly Example 1).

The components and concentration of the lipids, droplet size and solvent contributions can be varied to determine the ultimate effect on the lipid particle size. Further, the rate of forming lipid droplets, method of introducing droplets into the aqueous solution, the effect of agitating or not agitating the aqueous solution, temperature of the aqueous solution, electrolyte concentrations, etc. can all be investigated using routine experimentation. Thus, for a given droplet size, the effect of the presence of anionic or cationic lipids or surfactant resulting in a smaller liposome relative to the size obtained in the absence of these components can be investigated and optimized. Similarly, the solvent can be varied to investigate the role of solvent miscibility in water and diffusion rate of solvent into the aqueous phase on liposome size and characteristics. One skilled in the art would be able to employ routine experimentation to optimize the liposome or other lipid particle obtained for an intended use.

The aqueous solution may be agitated or otherwise mixed as the droplets are introduced using any known method such as, for example, a stir bar 18.

A. Generation of Droplets

As discussed above, one or more vesicle-forming lipids, and optionally oils and/or surfactants, are dissolved in a water miscible solvent, a water immiscible solvent, or mixtures thereof.

Droplets of the required size and/or lipid concentration can be produced using any appropriate device or method, such as using nebulization, atomization, focused acoustic ejection, electrospray, venturi mist generation, and the like. The droplet has a diameter of between about 0.01 microns and about 100 microns, although there is no lower limit to the size of the droplet that can be used. It will be appreciated that the lower limit of the droplet size is dependent upon the capabilities of the generation system. In a preferred embodiment, the droplets have a narrow size distribution. Preferably, the droplets have a diameter of less than about 10 microns, more preferably, less than about 5 microns. In preferred embodiments, the droplets have a diameter between about 0.1 microns and about 5 microns.

As stated above, the size of the liposomes formed by the method are primarily controlled by the size of the droplets and the concentration of lipid in the droplet. It will be appreciated that these parameters may be related. For example, a droplet having a diameter of 5 microns contains a volume of about 67 fL (femtoliters), and thus contains about 4×10⁷ lipid molecules, assuming a 1 mM solution of lipid in solution. A droplet of 2 microns in diameter has a volume of 4 fL, and thus contains about 2.4×10⁶ lipid molecules. A droplet having a diameter of 0.1 microns has a volume of 5×10⁻⁴ fL and thus contains about 300 lipid molecules. It will be appreciated that by choosing a certain concentration of lipid in the solvent (and optionally oils and surfactants and the like), one can make a droplet having a predetermined number of molecules that can be introduced in the aqueous solution as a discrete local concentration of lipid in water, thereby providing control over the size and composition of the lipid particles produced at the molecular level. Depending on the nature of the lipids (and other components) present, the solvent, the presence of surfactant in the aqueous solution or the solvent, the temperature and salt conditions in the aqueous solution, droplet sizes can be chosen to optimize the lipid particle produced.

In a preferred embodiment, each droplet is not more than about 1 microliter in volume. Preferably, the droplet volume is between about 10⁻⁴ fL and about 1 nL, and even more preferably, the droplet volume is between about 10⁻² fL and about 10 pL, although there is no lower limit to the size of the droplet that can be used.

Ink jet printing methods such as described in U.S. Pat. No. 4,697,195 to Quate, U.S. Pat. Nos. 4,751,529 and 4,751,530 to Elrod, and U.S. Pat. No. 6,596,239 to Williams have been shown to be capable of generating picoliter-sized droplets with an extremely tight size distribution. It will be appreciated that the droplets of the present invention can be formed using similar methods. According to the U.S. Pat. No. 6,596,239 patent, the size of the droplet can be controlled by modulating the frequency, voltage and duration of the energy source used to excite the acoustic emitter, generally a piezoelectric transducer. Droplet sizes are reported to be at least 1 micron in size.

The sizes of the lipid particles produced, as well as the sizes of the droplets introduced into the aqueous solution, can be determined using methods known in the art. A non-limiting list of methods for determining the sizes of the lipid particles and droplets includes: electron microscopy (freeze fracture, negative stain transmission EM, and scanning EM); submicron particle analyzer (e.g., Malvern Laser, Cascade Impactor, Coulter); field flow fractionation (FFF); capillary hydrodynamic fractionation (CHDF); laser diffractometry; and phase doppler analyser (PDA).

B. Focused Acoustic Ejection

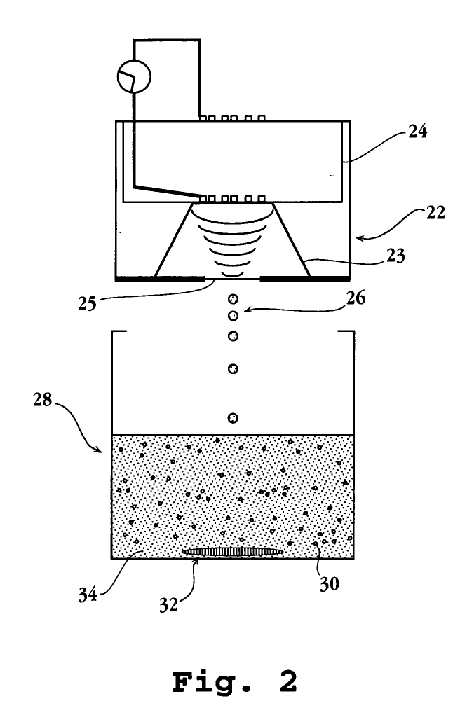

In another embodiment, shown in FIG. 2, the droplets 26 are formed by a focused acoustic ejector 22, as described in U.S. Publication No. 20030012892A1. Briefly, the device includes an acoustic ejector comprised of an acoustic radiation generator for generating acoustic radiation. The acoustic radiation is focused at a focal point within the reservoir containing the solvent and dissolved lipid 23 near the fluid surface 25. An acoustic ejector 24 is adapted to generate and focus the acoustic radiation so as to eject a droplet 26 of fluid from the fluid surface 25 into a collection vessel 28 containing an aqueous solution 34.

As described in Example 8, the lipids are dissolved in a solvent, preferably an alkanolic solvent such as ethanol, DMSO, ether or a halogenated hydrocarbon, to a desired lipid concentration. The lipid/solvent solution can also contain drugs, targeting ligands, lipopolymers and the like. To generate the solvent/lipid droplets, an acoustic lens array as described in U.S. Pat. No. 4,751,530 can be utilized. As noted above, the droplets are introduced into a collection vessel 28 containing aqueous solution 34 to form the lipid particles 30. Alternatively, the focused acoustic generation system 22 produces a mist that can be bubbled through the aqueous solution using a carrier gas, not shown. For example, a flow of nitrogen can be directed past the ejectors and the nitrogen containing ejected droplets can be bubbled through the aqueous solution.

The aqueous solution (optionally containing buffers, electrolytes, therapeutic agents, and the like) to be used for collecting the solvent/lipid droplets is preferably maintained at a temperature above the main phase transition temperature of the lipid used. It will be appreciated that the temperature can be varied according to the composition and the final product desired, especially for lipid particles other than liposomes.

When the droplets are introduced to the aqueous solution, the droplets are absorbed into the aqueous phase upon contact with the aqueous surface, and the solvent diffuses into the bulk aqueous phase. The lipid molecules from the droplet form liposomes or other lipid particles, depending on the components present in the droplets. Upon introduction of the droplet into the aqueous phase, the solvent diffuses out of the droplet into the aqueous phase and the lipids reform into bilayers or monolayers forming the lipid particles in the aqueous phase. When oil is present in the lipid solution, the oil droplet remains at the core of the droplet, and the acyl chains of the lipids spontaneously form a surface layer about the oil core. Additional excess lipids may form into concentric bilayers about the central oil droplet core. When nonionic surfactant is present, niosomes are formed. Depending on the proportion of oil, lipid, and surfactant present, liposomes, lipospheres, niosomes, emulsomes or emulsions are formed.

It will be appreciated that the aqueous reservoir may be in fluid communication with the reservoir of solvent/lipid to allow the direct capture of the droplets by the aqueous solution. The reservoirs may be in fluid communication using any suitable means including tubing connecting the reservoirs. In this embodiment, the droplets are generally introduced to the aqueous solution by bubbling the droplets through the aqueous solution using an inert carrier gas such as nitrogen. In this embodiment, losses such as may occur when ejecting droplets into the air or other gas phase prior to transport of the droplets into the aqueous solution may be prevented. However, it will be appreciated it is generally undesirable for the aqueous solvent to be introduced into the reservoir containing the lipid/solvent solution.

Focused acoustic ejection allows the ejection of droplets from 0.01 picoliters to 20 picoliters in volume (droplets having diameters as small as 2.7 microns), where the droplets can be produced at a rate of at least about 1,000,000 droplets per minute (U.S. Pat. No. 6,416,164 to Stearns). In other embodiments, focused acoustics have been used to generate droplets having a diameter of 5 to 10 microns (U.S. patent Publication No. 20020077369). The focused acoustic energy is generally used to generate liquid droplets whose diameter is on the order of the acoustic wavelength. In other words, droplet sizes are typically on the order of the wavelength of the bulk acoustic wave propagating in the solvent solution. This wavelength may be determined by dividing the velocity of sound for bulk wave propagation in the solvent by the frequency of the bulk acoustic wave. Thus by increasing frequency, droplet size can be reduced. A RF drive frequency exceeding 300 MHz typically results in the generation of droplets smaller than 5 microns in diameter.

In another embodiment, capillary wave generation is used to generate the droplets as described in U.S. Pat. No. 6,622,720. When generating capillary wave-driven droplets, the principle mound does not receive enough energy to eject a droplet. Instead, as the principle mound decreases in size, the excess liquid is absorbed by surrounding capillary wave crests or side mounds. These wave crests eject a mist corresponding to droplets 26. In order to generate capillary action droplets instead of focused, single ejection droplets, each ejector transducer generates shorter pulse widths at a higher peak power, typically on the order of 5 microseconds or less at a peak power of approximately one watt or higher per ejector. Capillary action may be used to create smaller droplets at lower frequencies. The diameter of capillary generated droplets is similar in magnitude to the wavelength of capillary waves.

Liposome size can be measured by a submicron particle analyzer (e.g., Coulter N4MD). The frequency of the acoustic power generator can be adjusted to produce droplets in the range of 0.001 fL to 50 pL to achieve the desired liposome size. For parenteral injection, the final mean size should be in the range of 80-200 nm. If the final liposome size is larger than desired for a particular droplet size, the lipid concentration can be appropriately reduced.

In another embodiment, a plurality of ejectors and reservoirs containing the solution of lipids can be provided, not shown. An array of focused acoustic ejectors can be positioned beneath a microtiter plate for ejection of microdroplets of lipid in solvent directly into the aqueous phase above or below. The microtiter plate containing the lipid solution can be in sealed contact with the aqueous phase. Because of the small size of the openings of the reservoirs of the microtiter plates, there is no mixing between the aqueous phase and the solvent phase. Alternatively, a less miscible (or more or less dense) solvent can be used to prevent mixing of the aqueous and solvent phases prior to introduction of the droplets into the aqueous solution. Microdroplets are ejected using focused acoustic ejection directly into the aqueous phase, which is being preferably stirred or otherwise agitated using mixing means 32 to allow rapid mixing of the ejected droplets in the aqueous solution. Alternatively, as discussed above, solvent/lipid mists can be directed into the aqueous solution using a carrier gas or using ejected droplet trajectories. In addition, each ejector can be activated at a high frequency so as to produce droplets at a rapid rate.

C. Nebulization and Atomization

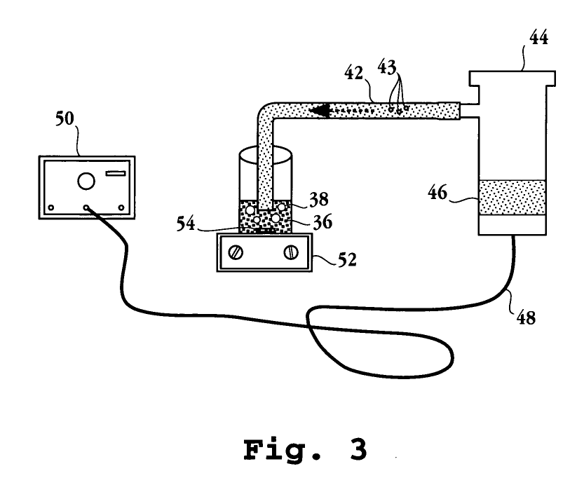

In another embodiment, as shown in FIG. 3, the droplets 43 are formed by a nebulizer or atomizer 44. Nebulizers and atomizers can produce droplets of varying sizes, including droplets in the range from submicrons to hundreds of microns in diameter, typically in the range of 1 to 10 microns in diameter. For purposes of the present method, droplets having diameters in the ranges of 0.01 microns to about 100 microns, and more preferably from about 0.1 microns to about 10 microns are generated. Nebulizers are generally of two types: jet (or pneumatic) small-volume nebulizers, and ultrasonic nebulizers. Jet nebulizers are based on the venturi principle, whereas ultrasonic nebulizers use the converse piezoelectric effect to convert alternating current to high-frequency acoustic energy.

In one embodiment, a compressed air nebulizer 44 (e.g., AeroEclipse, Pari L. C., the Parijet, Whisper Jet, Micronebe®, Sidestream®, Acorn II®, Cirrus® and Upmist®) generates droplets 43 as a mist by shattering a liquid stream with fast moving air supplied by tubing 48 from an air pump 50. Droplets that are produced by this method typically have a diameter of about 2-5 μm.

In another embodiment, an ultrasonic nebulizer that uses a piezoelectric transducer to transform electrical current into mechanical oscillations is used to produce aerosol droplets from the lipid/solvent solution. These droplets have a diameter in the size range of 1 to about 5 microns. It will be appreciated that any suitable ultrasonic nebulizer may be used as exemplified by the Aeroneb® Nebulizer (Aerogen, Inc., Mountain View, Calif.), MicroNeb III, Pari Plus and Pari Star (for generating droplets less than 5 microns, Pari, Starnberg, Germany), Ventstream, Omron U1, UMIST nozzle, airbrush nozzle, AeroEclipse, the sonic spray nebulizer as described by Huang, et al., (1999) Anal. Sci. 15:265 (1 micron droplets), Skylark ultrasonic nebulizer (3-8 microns, Taiwan), disposable medical nebulizer (Raindrop; Puritan Bennett, Lenexa, Kans., having a diameter of 3.2 microns (+/−1.9) as determined by an Andersen cascade impactor).

Droplets of a desired size can be produced by selection of a nebulizer, jet or ultrasonic, that produces droplets in the range desired. It is further within the ability of one skilled in the art to modify the nebulizer to adjust the diameter of the droplets produced. In one embodiment, a droplet impactor plate is used to remove droplets above a given diameter produced by any particular nebulizer, atomizer or other droplet source, if droplets are produced above a threshold desired size, and the material can be recycled. In addition, droplets can be produced having a desired size range by use of a nebulizer and further selecting an appropriate nozzle size. It will be appreciated that a plurality of nozzles can be used to enhance the rate of production of droplets. It will further be appreciated that a droplet impactor plate can be employed to remove droplets above a given diameter, if droplets are produced above a threshold desired size.

Nebulization of the solvent/lipid solution 46 results in a fine spray or vapor of droplets 43. The nebulized lipid/solvent mist 43 is directed toward the aqueous solution 36 using suitable mechanisms, such as tubing 42. In another embodiment, the lipid/solvent mist 43 is bubbled 38 through the solution 36 for capture of the droplets, as shown in FIG. 3. The bubbling action may provide agitation of the aqueous solution as well, which although not necessary for formation of the lipid particles, can increase the efficiency of mixing and speed as well as improve reproducibility of the process. In another embodiment, the aqueous solution can be agitated using a conventional stirring device 52 and stir bar 54.

The aqueous solution (containing buffers, electrolytes, and the like) to be used for collecting the solvent/lipid droplets is preferably maintained at temperature above the main phase transition temperature of the lipids to be included in the lipid particle. The droplets can be introduced by any method known in the art. In one embodiment, the aqueous solution can be agitated in a vessel which is specially designed for maximum exposure of the liquid surface area by running the solution through a honey-comb like matrix (similar to in the design of a car radiator) made of stainless steel sheets. The stream of solvent/lipid mists is directed towards the aqueous vessel and the droplets are absorbed upon hitting the aqueous surface. Lipid molecules contained in the solvent droplet will form liposomes or other lipid particle in the aqueous solution according to the components present and the aqueous conditions. The liposome or lipid particle size can be measured by a submicron particle analyzer.

The solvent, lipid composition, temperature and aqueous solution can be varied to determine the effect of these parameters using routine experimentation. Nebulizers producing droplets in different ranges can be tested until the desired liposome or other lipid particle size is achieved. For example, for parenteral injection, the final mean size should be in the range of 80-200 nm. If the final lipid particle size is larger than desired, the lipid concentration can be appropriately reduced.

As described in Example 1, liposomes having a suitable size for intravenous injection (diameter of 166±6 nm) were formed by nebulizing an ethanol/POPC solution and introducing the droplets to DI water. As described in Example 2, modification of the solvent and lipid parameters (by using ether as the solvent and having a lipid concentration of 20 mg/mL of POPC), the liposomes formed had a diameter of 1160±140 nm. Thus, modification of the lipid concentration and/or use of other solvents can be used to modulate and target the liposome size.

As discussed in Examples 3 and 4, liposomes were formed from droplets generated by nebulization. By these methods, liposomes having a diameter of 166 and 223 nm were formed. Accordingly, the liposomes of about 100-150 nm and about 200-250 nm were formed by modulation of the lipid concentration. The trapped volume of the liposomes in the experiments was 15.5 mL/mmole lipid and 11.4 mL/mmole lipid, respectively. These values for trapped volume are higher than for liposomes prepared using most prior art methods, suggesting a decrease in the amount of multilamellar liposomes present or a decrease in size heterogeneity. The trapped volume for extruded liposomes is typically 1-2 mL/mmole lipid, for liposomes prepared using ethanol or ether injection, trapped volumes are in the range of 5-10 mL/mmole, and for sonicated liposomes, the trapped volumes are in the range of 0.2 to 0.5 m/mmole (Zhang, et al., Liposomes in Drug Delivery, in Polymeric Biomaterials, 2^{nd} edition, S. Dumitriu, Ed., Marcel Dekker, Inc., New York (2001)).

As described in Example 2, preparation of liposomes using droplets generated by nebulization was compared with direct injection of the ether/POPC solution into an aqueous solution. Liposomes having a mean diameter of 1160±140 nm as measured by a Coulter submicron particle sizer were formed by the droplet generation method. In contrast, when the ether/lipid solution was slowly injected directly into deionized water, no liposomes were formed. Thus, formation of lipid particles using the droplet generation method may proceed under conditions that would not otherwise be amenable to formation of lipid particles.

It will be appreciated that additional technologies are available to produce droplets suitable for use in the present methods and formulations such as vibrational frequency as described in Example 7. Electrospray (and nanospray) technologies can be used to generate droplets of suitable size. Conventional electrospray produces droplet sizes of less than 10 microns, and at higher voltages, even smaller droplets can be produced. A venturi mist generator has been reported to produce droplets that peak at 0.43 microns, or about 0.04 fL (U.S. Pat. No. 6,511,718).

One skilled in the art will recognize that residual solvent may remain present in the lipid particle once formed. Excess solvent present can be removed if desired, e.g., by dialysis or diafiltration for water-miscible solvents such as ethanol and DMSO, and by vacuum evaporation for non-water-miscible solvents such as ether and chloroform. However, solvent removal may not be necessary, depending on the amount of residual solvent and the acceptability of the residual solvent in the formulation.

One skilled in the art will appreciate that the ratio of lipids and optionally oils and surfactants in the preparation has an effect on the form of the lipid particle prepared, and determines whether a liposphere, emulsion, liposome, niosome or emulsome is prepared. Likewise, the temperature and aqueous conditions, such as pH and ionic strength, can also have an effect on the lipid particles and liposomes prepared using the methods described herein, and one skilled in the art can investigate the effects of varying solvent, lipid content, lipid concentration, temperature, and aqueous conditions using no more than routine experimentation. As described in Examples 5 and 6, modification of the lipid concentration and inclusion of triolein, an oil, results in formation of lipospheres or emulsomes, respectively.

In another aspect, the method may be used to prepare lipid particles having a predetermined diameter or size distribution. Similar to above, droplets of solvent/lipid are generated and introduced into an aqueous solution to form the lipid particles. Additionally, droplets having a different solvent/lipid composition (such as different lipid or solvent or different solvent/lipid concentration) are generated and introduced into an aqueous solution to form lipid particles. The lipid particles formed from the two solvent/lipid solutions can be compared based on factors such as diameter, volume, etc. In this manner, the conditions for preparation of lipid particles having desired properties may be determined. It will be appreciated that a similar technique may be employed to investigate other factors affecting the lipid particles such as the size (diameter and/or volume) of the droplets generated.

It is to be understood that while the invention has been described in conjunction with the preferred specific embodiments thereof, that the description above as well as the examples that follow are intended to illustrate and not limit the scope of the invention. The practice of the present invention will employ, unless otherwise indicated, conventional techniques of organic chemistry, polymer chemistry, biochemistry and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

### IV. EXAMPLES

The following examples illustrate but are in no way intended to limit the invention.

In the following examples, efforts have been made to ensure accuracy with respect to numbers used (e.g., amounts, temperature, etc.) but some experimental error and deviation should be accounted for. Unless indicated otherwise, temperature is in degrees centigrade (° C.) and pressure is at or near atmospheric pressure. All solvents were purchased as HPLC grade, and all processes were routinely conducted under standard atmosphere unless otherwise indicated. Unless otherwise indicated, the reagents used were obtained from the following sources: phospholipids, from Avanti Polar Lipids, Inc. (Birmingham, Ala.); organic solvents, from Aldrich Chemical Co. (Milwaukee, Wis.); and gases, from Matheson (Seacaucus, N.J.).

Particle sizes were measured using a Coulter submicron microsizer (Model N4MD).

### Example 1

### Preparation of Liposomes Using Nebulizer Generated Droplets

0.57 g of POPC (NOF Corp) was dissolved in ethanol (absolute ethyl alcohol USP, lot 99F15QA, MPER Alcohol and Chem. Co.) in a 5 mL scaled flask. The final lipid concentration was 110 mg/mL. Two milliliters of the POPC:ethanol solution was loaded into a PARI LC STAR nebulizer (Pari Respiratory, Starnberg, Germany, model 22F51) to generate droplets of the POPC:ethanol solution. The air flow for aerosol generation was generated using a DURA-NEB® 3000 (Pari Respiratory) portable aerosol system coupled to the bottom of the nebulizer through tubing.

The nebulized droplets were introduced to a 100 mL glass beaker containing 45 mL of deionized water (DI) through 0.5 cm diameter, size 18 flexible tubing connected to the outlet of the nebulizer with continuous stirring. When the air pump was turned on, the ethanol mist bubbled through the water. The water slowly became translucent, indicating that liposomes were being formed. The liposome size was determined to be 166±6 nm (n=3) as measured by a Coulter submicron particle sizer.

### Example 2

### Preparation of Liposomes Using Ether Solvent and Generation of Droplets With Nebulizer

POPC was dissolved in anhydrous ether to a final concentration of 20 mg/mL. Ten milliliters ether solution in 2 mL increments was nebulized into 50 mL DI water as described in Example 1. The deionized water was maintained at 40° C. with continuous stirring. After the air pump was turned on to start the nebulization, the water solution quickly became translucent, indicating liposomes were being formed. The liposome mean diameter was determined to be 1160±140 nm as measured by a Coulter submicron particle sizer.

As a comparison, 0.5 mL of the ether/lipid solution was slowly injected into 5 mL deionized water at 40° C. A thick, chunky gel formed in the upper part of the solution, and no liposome formation was apparent.

### Example 3

### Encapsulation Efficiency of Liposomes Formed with Nebulization

490 mg of POPC was dissolved in 25 mL ethanol to a final lipid concentration of 9.6 mg/mL. The lipid/ethanol solution was nebulized using a device as described in Example 1 into 30 mL of DI water containing 0.6 mg/mL of dextran fluorescein, a fluorescent dye (10,000 MW, Molecular Probes, D-1821, lot 9A) in a scaled 50 mL volume cylinder at room temperature. The cylinder was used to increase the exposure of the water to the lipid/ethanol mists. 10 mL of the lipid/ethanol solution was nebulized and introduced into the cylinder. After nebulization, the total volume in the cylinder was about 35 mL. The final lipid concentration in the aqueous suspension was determined to be 3.35 mg/mL as assayed by phosphorous content. Thus, the efficiency of capture of the nebulized lipid by the aqueous solution was nearly 60%. The liposome diameter was 166±4 nm as measured by a Coulter submicron particle sizer. On day 4, the liposome diameter was 188±5 nm as measured by a Coulter submicron particle sizer.

In order to determine encapsulation efficiency of the dye by the liposomes, the unentrapped dye was separated from the liposomes by diafiltration (cartridge: A/G Tech Corp., UFP-100-E-MM01A, 100,000 NMWC, 1 mm, 16 cm²). Measurement of the fluorescent intensity of the pre- and post-diafiltration samples indicated an encapsulation efficiency of 6.6%. Given the lipid concentration of 4.26 mM, the trapped volume of the liposome was calculated to be 15.5 mL/mmole.

### Example 4

### Encapsulation of a Fluorescent Dye in Liposomes Generated with Nebulization

650 mg POPC was dissolved in 25 mL ethanol to a final lipid concentration of 26 mg/mL. The lipid/ethanol solution was nebulized using a device as described in Example 1. The nebulized droplets were introduced into 30 mL DI water containing 6.4 mg/mL of HPTS, a fluorescent dye (Molecular Probes Inc. H348 lot: 0181-2) in a scaled 50 mL cylinder at room temperature. In this experiment, the tube introducing the droplets was pinched to reduce the flow of gas, which may have affected the delivery of droplets and/or the droplet size into the aqueous solution. A total of 3.5 mL lipid-ethanol solution was nebulized and introduced into the DI water. After nebulization and introduction, the total volume in the cylinder was about 32 mL. The final lipid concentration in the aqueous suspension was determined to be 0.64±0.16 mg/mL (0.81±0.2 mM, n=3) as assayed by phosphorous content. This translates to a value of 24% for the efficiency of capture of the nebulized droplets by the water. The liposome diameter was determined to be 223±6 nm (n=3) as measured by a Coulter submicron particle sizer.

In order to determine the dye encapsulation efficiency, the unentrapped dye was separated from the liposome entrapped dye by passing 200 microliters of the lipid suspension through a Sephadex G50 (Pharmacia) column (30 cm long×0.5 cm diameter) and liposomes were eluted with saline (0.9% NaCl). A total of 40 fractions (25 drops/fraction) were collected. Fractions 4-8 containing the liposomes were pooled for a total volume of 3.15 mL; and fractions 24-35 containing the unentrapped dye totaled 7.5 mL in volume. Measurement of the fluorescence intensity of the two pooled fractions indicated a trapping efficiency of 0.92%. The recovery from the G50 column was 100% (104% actual). Given the lipid concentration of 0.81±0.2 mM, the trapped volume was calculated to be 11.4±2.9 mL/mmole.

### Example 5

### Preparation of Lipospheres

100 mg of POPC and 200 mg of triolein are dissolved in 25 mL DMSO/ethanol (1:1 v/v). The lipid/solvent solution is nebulized using a device as described in Example 1 and introduced into 30 mL DI water contained in a scaled 50 mL cylinder at room temperature. A total of 4.0 mL lipid solution is nebulized and introduced into the water to form lipospheres. The concentrations of lipid and triolein are determined by HPLC, and the diameter of the lipospheres is measured using a Coulter N4MD submicrosizer.

### Example 6

### Preparation of Emulsomes

200 mg of POPC and 100 mg of triolein are dissolved in 25 mL DMSO/ethanol (1:1 v/v). The lipid/solvent solution is nebulized using a device as described in Example 1 and introduced into 30 mL DI water contained in a scaled 50 mL cylinder at room temperature. A total of 4.0 mL lipid solution is nebulized and introduced into the water to form emulsomes. The concentrations of lipid and triolein are determined by HPLC, and the diameter of the particles is measured using a Coulter N4MD submicrosizer.

### Example 7

### Preparation of Liposomes Using Vibrational Frequency Generated Droplets

Ten grams of HSPC/Cholesterol/mPEG2000-DSPE (55:40:5) is dissolved in 100 mL ethanol to a final lipid concentration of 0.1 g/ml. The droplets are generated as a solvent/lipid mist by vibrational frequency using a device similar to that described in U.S. Pat. No. 6,405,934 to Hess. Briefly, the device uses vibrating means to apply a frequency vibration to the solvent/lipid solution thereby generating the liquid droplet spray. The liquid droplet spray is then ejected through an outlet. The droplet size is inversely proportional to the excitation frequency as of a particular frequency and pressure. The stream of solvent/lipid mist is directed towards a vessel of containing an aqueous solution. The droplets are absorbed upon contacting the aqueous surface and liposomes are formed in the aqueous solution. The aqueous solution is maintained at temperature above the main phase transition temperature (60-65° C.). Liposome size is measured by a submicron particle analyzer such as a Coulter N4MD submicrosizer. The frequency of the vibration is adjusted to produce droplets in the range of 50 fL to 5 pL until the desired liposome size is achieved, preferably liposomes having a diameter of 50-200 nm.

### Example 8

### Preparation of Liposomes Using Focused Acoustics Generated Droplets

Ten grams of HSPC/Cholesterol/mPEG2000-DSPE (55:40:5) is dissolved in 100 mL ethanol to a final lipid concentration of 0.1 g/ml. The droplets are generated as a solvent/lipid mist by focused acoustic ejector using a device as depicted in FIG. 2. Briefly, the device generates acoustic radiation using a suitable power source such as a RF power source. The ejector focuses acoustic radiation at a focal point near the fluid surface of the solvent/lipid reservoir thereby to eject a droplet from the device. The droplet spray is introduced into a reservoir containing an aqueous solution such as DI water. The droplets are absorbed upon contacting the aqueous surface and liposomes are formed in the aqueous solution. Liposome size is measured by a submicron particle analyzer such as a Coulter N4MD submicrosizer. The frequency of the radiation is adjusted to produce liposomes having a diameter in the range of 50-200 nm.

1. A method for preparing lipid particles, comprising:
producing discrete droplets of vesicle-forming lipids in a solvent, said droplets having a diameter and a volume;
introducing said droplets into an aqueous solution; and
forming lipid particles suitable for in vivo administration.

2. The method of claim 1, wherein said lipid particle is a liposome.

3. The method of claim 1, wherein the lipid is selected from the group consisting of distearoyl phosphatidyl choline, distearoyl phosphatidyl ethanolamine, and hydrogenated soy phosphatidyl choline.

4. The method of claim 1, further comprising:
including a therapeutic agent in at least one of the solvent or the aqueous solution.

5. The method of claim 4, wherein said therapeutic agent is an anthracycline antibiotic.

6. The method of claim 5, wherein said anthracycline antibiotic is selected from the group consisting of daunorubicin, doxorubicin, mitoxantrone, and bisantrene.

7. The method of claim 1, further comprising:
including a lipopolymer in said droplet.

8. The method of claim 7, wherein said lipopolymer is selected from the group consisting of polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, and polyaspartamide.

9. The method of claim 8, wherein said lipopolymer is polyethylene glycol chains having a molecular weight of between about 500 Daltons and about 10,000 Daltons.

10. The method of claim 7, further comprising a ligand attached to the distal end of at least a portion of said lipopolymers.

11. The method of claim 1, further comprising a ligand attached the polar head group of at least a portion of the vesicle-forming lipids.

12. The method of claim 1, wherein the concentration of lipid in each droplet is between about 0.1 mg/mL and about 1 g/mL.

13. The method of claim 1, wherein the concentration of lipid in each droplet is between about 1 mg/mL and about 100 mg/mL.

14. The method of claim 1, wherein the droplet volume is between about 10⁻⁴ fL and about 1 nL.

15. The method of claim 1, wherein the droplet volume is between about 10⁻² fL and about 10 pL.

16. The method of claim 1, further comprising:
including at least one of a cationic lipid, an anionic lipid, a surfactant, a marker, an oil, or a pharmaceutical excipient in said solvent.

17. The method of claim 1, further comprising:
applying a focused acoustic radiation at a focal point near a surface of the solution prior to and/or during said introducing.

18. The method of claim 1, said introducing step further comprising:
providing a plurality of ejectors such that a plurality of droplets can be ejected from a plurality of solvent reservoirs containing said lipids and solvent.

19. The method of claim 1, wherein said discrete droplets are produced as a mist and said introducing step comprises:
directing the mist of droplets into contact with the aqueous solution.

20. The method of claim 19, wherein the mist of droplets is generated by a system selected from the group consisting of a nebulizer, an atomizer, a venturi mist generator, a focused acoustic ejector, and an electrospray device.
